(19) United States
(12) Patent Application Publication
(10) Pub. No.: US 2014/0066692 A1
(43) Pub. Date: Mar. 6, 2014

(54) BABY BLANKET

(71) Applicant: Serge V. Monros, Santa Ana, CA (US)

(72) Inventor: Serge V. Monros, Santa Ana, CA (US)

(21) Appl. No.: 13/603,142

(22) Filed: Sep. 4, 2012

(51) Int. Cl. A61M 21/02

(52) U.S. Cl. 600/28

(57) ABSTRACT

An amniotic infant blanket provides a feeling of fluid warmth coupled with the audible sounds of a human heartbeat. Behind a head or face portion of the blanket, a pocket is provided into which a bladder may be removably inserted. The pocket may be sealed by a zipper and has an aperture through which a hydrophone wire may pass. Likewise, the hydrophone wire may pass through the wall of the bladder without creating a leak therethrough. The bladder is filled with lukewarm tap water and the hydrophone connected to a playback unit that transmits signals that are transformed by the hydrophone into heartbeat sounds with which the infant is familiar.

### FIELD OF THE INVENTION

This invention relates to infant blankets and, more particularly, to an infant blanket that soothes the infant by offering it a warm fluid cushion within which heartbeat sounds may be heard.

### DESCRIPTION OF THE RELATED ART

Small blankets have frequently been used to provide warmth and comfort for infants during the first few years of life. In order to provide the infant with a more enjoyable blanket, improvements in infant blankets have arisen over the years. Generally, simulation of the amniotic environment experienced by the infant prior to birth tends to soothe and make quiet the infant so that both it and its parents may relax.

Several attempts have been made previously in order to provide infants or young children with a warm and soothing environment. Some attempts have been made in the following listed art for which brief descriptions are given.

B. J. Fornarelli. U.S. Pat. No. 5,205,811

This patent is directed to a baby blanket with a heartbeat simulator. The infant blanket is shown in FIG. 1 and is formed from a padded material 12 with a pouch 13 with access by means of zipper 20. FIG. 2 shows the device that has been opened to receive heart simulator 18. The simulator is cased within a foam form which is placed into the blanket recess and is activated when the baby is placed into the area overlying the pouch portion.

L. Belkin, U.S. Pat. No. 3,292,611

This reference describes an infant mattress that includes both a heating device and a mechanism to provide comfort to the child by means of a mechanical clock device. The ticking device is meant to convey to the child a feeling that resembles the mother's heartbeat. The ticking device 26 is placed directly beneath the child and may be operated by a winding or electrical mechanism. Heating unit 32 is used to provide heat to the infant.

B. A. Reich. U.S. Pat. No. 4,790,042

This patent is directed to a baby comforter which provides the child with a feeling of warmth and gives the feeling of being held by another individual. Referring to FIGS. 1-3, the apparatus 20 includes a body portion 22 which is constructed from a generally rectangular piece of soft, washable, durable cloth. A length 36 of one of the complementary forms of Velcro-type hook-and-eye material is attached adjacent to edge 28 on face 32. The cloth 23 can be folded in a manner of a portfolio to form a pocket 42 for receiving a hot water bottle 44.

L. J. Clute. U.S. Pat. No. 5,357,642

This patent describes a support pillow which is used to provide an infant with a generated sound that simulates that experienced by the infant when in the womb. Referring to FIG. 1, the device includes a chamber 136 with access by means of slit 138 designed to receive audio emitter 140. The audio emitter 140 is designed to provide sounds that simulate a mother's heartbeat as well as other sounds such as music. The sound that is projected through the phone pad by the emitter also creates a vibration which can be felt by the infant using the device. This vibration has been found to be somewhat comforting and, along with the sounds produced, tends to provide comfort to the infant utilizing the pillow device.

R. Cohen. U.S. Pat. No. 4,969.867

The apparatus described in this reference is used in the form of a blanket to promote sleep. The blanket may be seen in FIG. 1 and comprises a sandwiched structure wherein sound transducers 4 are distributed between layers 2a and 2b. The transducers 4 produce both sound and compressional waves so that the infant may sense a vibration as well as hearing sounds. One embodiment includes a signal generator which is intended to simulate the human heartbeat.

B. L. Cummins. U.S. Pat. No. 4,066,072

This patent is directed to a comfort cushion for infants, which is designed to simulate the temperature, sound, and other stimuli which a child would experience within the mother's womb. The system comprises a fluid-filled, flexible mattress 11 with a pulsating fluid pump 12 as well as other elements. Since the device uses a flowing fluid as well as operating valves, sounds and vibrations are emitted and the infant is provided with an environment that simulates prenatal experiences. A means is also provided to control the temperature so that the infant may be kept warmed to a comfortable temperature level.

Despite the developments in previous devices related to infant blankets, sleeping surfaces, and heartbeat-sound generation, no previous devices are known where the heartbeat emanates from within a fluid-filled sac, or bladder, as would be present in the pre-birth amniotic environment. Therefore, it can be seen that greater infant comfort and care could be delivered by the provision of a pleasing, soothing, and comfortable infant blanket that lulls the infant into a relaxed state by providing some greater and more direct reminders of its previous amniotic environment.

### SUMMARY OF THE INVENTION

The present invention lies in a comforting and soothing infant blanket that uses a hydrophone to transmit heartbeat-like sounds to the adjacent infant through a fluid-filled sac, or bladder. A soft, outer shell provides the surface of the blanket and may be machine washable for easy cleaning. Generally, the soft, outer shell is in the shape of a sheep but may also have a shape of other friendly animals. The surface of the outer shell is generally soft and absorbent and possibly constructed of soft cotton, terry cloth, and/or fleecy sheepskin.

In the embodiment resembling a sheep, the amniotic infant blanket of the present invention may have a silk-screened sheep face attached to a portion resembling the sheep's head. Behind the sheep's head, an opening in the soft, outer shell may lead into a chamber immediately behind the silk-screened face or other portions of the blanket front. Inside the chamber, a non-leaking bladder, or sac, may be provided in which warm fluid such as water may held. The warm bladder will remind the infant of its pre-birth environment. Such reminiscence is enhanced by the use of a hydrophone residing within the bladder.

The hydrophone transmits sound into the surrounding fluid of the bladder and onto the infant so the infant may hear such sounds. Preferably, such sounds are those of the human heartbeat as would be heard by the infant in its pre-birth environment. A wire leading out from the hydrophone passes through the wall of the bladder. The interface between the wire and the bladder is well sealed to prevent any leakage of fluid therethrough. The wire of the hydrophone leads to a digital playback device having a digital recording of the heartbeat. When switched on, the digital playback unit continuously repeats the heartbeat sound for the benefit of the infant. In order to allow easy removal of the bladder from the chamber, a plug-and-socket combination may be used to disconnect the hydrophone from the digital playback unit. If the materials used in the bladder and the hydrophone can withstand such treatment, placement of the fluid-filled bladder in a microwave oven for fluid heating is contemplated.

Other features and advantages of the present invention will become apparent from the following more detailed description, when taken in conjunction with the accompanying drawings, which illustrate, by way of example, the principles of the invention.

### OBJECTS OF INVENTION

It is an object of the present invention to provide a blanket that soothes and comforts an infant.

It is an object of the present invention to provide a blanket that soothes and comforts an infant by providing a soft, cuddly surface covering a warm fluid bladder through which the sounds of a heartbeat may continually be heard.

It is another object of the present invention to provide an amniotic infant blanket that is visually attractive.

It is yet another object of the present invention to provide an amniotic infant blanket that is washable and durable.

These and other objects and advantages of the present invention will be apparent from a review of the following specification and accompanying drawings.

### BRIEF DESCRIPTION OF THE DRAWINGS

The accompanying drawings illustrate the invention. In such drawings:

FIG. 1 shows a front plan view of the amniotic infant blanket in a sheep-design embodiment.

FIG. 2 shows a back plan view of the amniotic infant blanket of FIG. 1.

FIG. 3 shows a cross section view of the amniotic infant blanket of FIG. 1 taken along line 3-3.

FIG. 4 shows the bladder-hydrophone-player configuration with the cap of the bladder removed as for fluid flow through the open aperture.

### DETAILED DESCRIPTION OF THE PREFERRED EMBODIMENTS

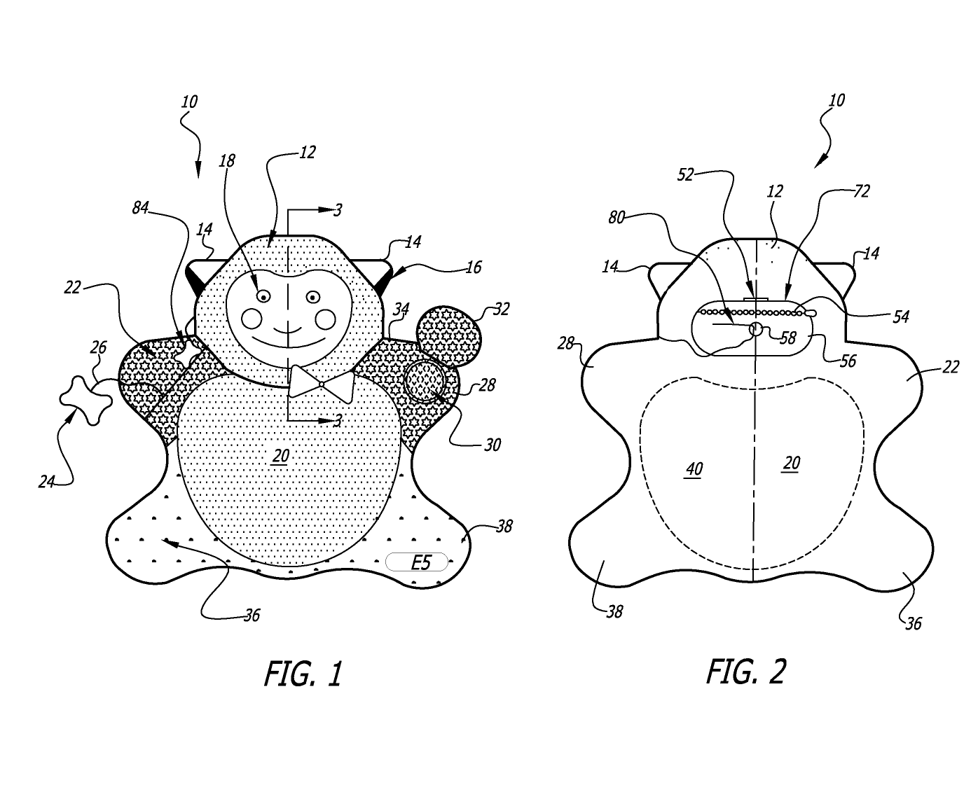

Referring to FIG. 1, the amniotic infant blanket 10 of the present invention is generally flat when spread out and may be configured in the shape of a stylized sheep having a head 12 with ears 14. The ears 14 may have colored cotton 16 placed therein, one such color being pink. The head 12 may have a face 18 resembling a stylized face of a sheep.

The head 12 is attached to a torso, or body section, 20 from which four appendages, the arms and the legs, are attached.

As shown in FIG. 1, the blanket's right arm 22 is generally round in nature and may have attached thereto a toy lamb 24 by a string, or cord, 26. The toy lamb may be used for play, teething, or the like by the infant. The left arm 28 of the sheep may hold a mirror 30 into which the infant may gaze and see itself. The mirror 30 is generally covered by a fabric or other material flap 32 and may allow a parent or other individual to play games of peekaboo with the infant as its face and eyes are turned toward the mirror. Such games sometimes provide endless fascination for infants. A bow tie 34 may decorate the juncture between the torso 20 and the head 12.

The right foot 36 and the left foot 38 may arise as lobes emanating from the torso 20. The same may be similarly true for the right and left arms 22, 28.

The front and back portions of the blanket 10 are made of an outer shell in the shape of a stylized sheep. As shown in FIG. 1, the torso 20 and outer portions of the head 12 are made of a sheepskin material that is fleecy, warm, and soft. The same is true for the outer portion of the ears 14 with the inner portions having a surface of pink cotton. The arms 22, 28 of the blanket 10 may be made of a cotton material having a paisley design. The right and left legs 36, 38 may be made of soft cotton such as terry cloth or the like.

The back 40 of the blanket 10 may also be made of soft cotton or other materials. Other materials such as polyester and wool may also be used in the present invention. Other materials will suggest themselves to those constructing the blanket of the present invention in order to create a soft, warm, and comfortable environment for an infant. The outer shell 50 of the blanket 10 is durable, washable, and stain resistant in light of its use with an infant.

Infants are well-known for creating and causing many spills, stains, and messes; and the same should be easily removed as by washing the blanket 10 of the present invention. Likewise, such washing should serve to sterilize and make biologically safe the blanket for the infant as they are wont to chew upon the various parts of the blanket such as the toy lamb 24. The blanket 10 of the present invention may be approximately 28 inches in height and 30 inches in width in order to allow an infant to comfortably lie upon the blanket 10. As shown in FIG. 2, in the back of the head 12 of the blanket 10 is a chamber 52 which is sealed by a zipper or other means 54. The chamber 52 provides a space into which a bladder, or sac, filled with warm fluid (described in more detail below) may fit and provide soothing warmth and comfort to the infant lying upon the blanket 10. The bladder pocket 52 may be of variable size and depth, but the present invention contemplates the bladder pocket as resting behind the silk-screened face 18 of the blanket 10. The zipper 54 allows the bladder pocket 52 to be opened and closed at the parent's convenience. A back flap 56 covering the bladder pocket 52 may have a hole, or aperture, 58 through which a wire may pass, connecting a hydrophone to playback means.

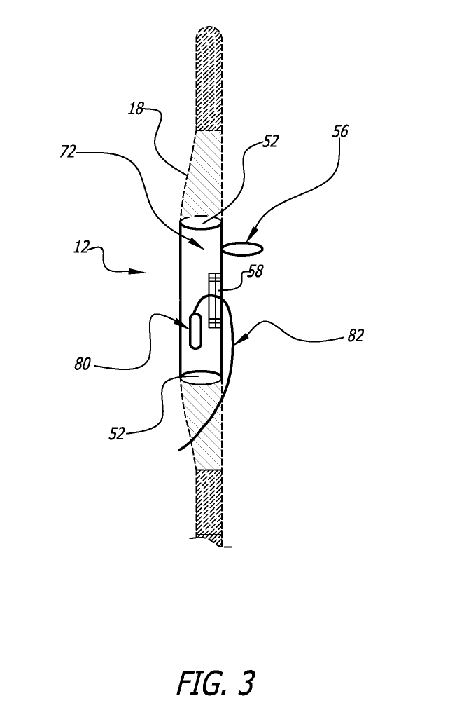

In FIG. 3, the head 12 of the sheep is shown in a side view taken along lines 3-3 of FIG. 1. Here, it is shown that the bladder-hydrophone assembly 70 has a relatively low profile, such that the head of the infant is not propped up too far. The hydrophone 80 is placed within the bladder 72, such that the hydrophone 80 is free-floating. The wire 82 extends from the hydrophone 80, through the bladder 72 and out through the hole or aperture 58 for connection to a playback unit 84 (not shown). The bladder-hydrophone assembly 70 fits within the bladder pocket 52 and is secured therein by the back flap 56.

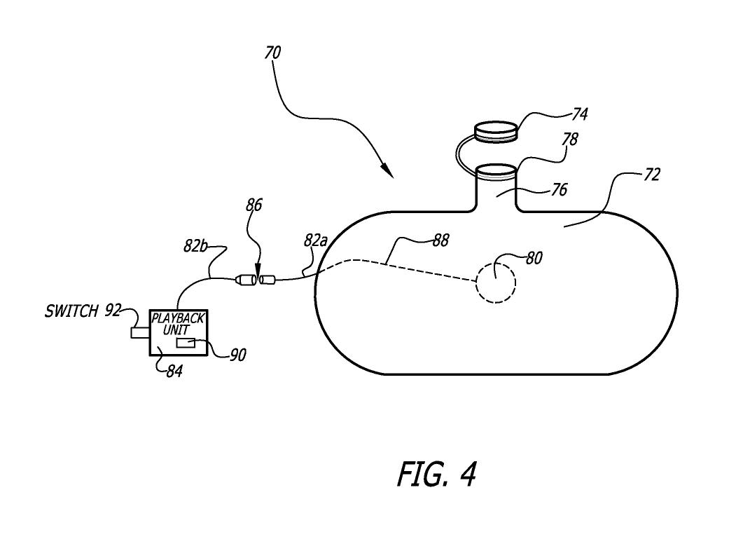

Referring now to FIG. 4, the bladder-hydrophone assembly 70 has a bladder 72 with a removable cap 74. The removable cap 74 detachably attaches to a neck or other protuberance 76 that has a threaded aperture end 78. Threaded aperture end 78 threadably attaches to the removable cap 74 by threads (not shown) along the interior of the removable cap 74. The neck 76 provides means by which the bladder 72 may be filled with warm fluid, such as warm tap water, and emptied when the fluid becomes cold or when it is otherwise desired.

The hydrophone 80 is of design and construction known in the art and resides within the interior of the bladder 72. The hydrophone 80 is connected by a wire 82 to a playback unit 84. The playback unit 84 is also of known design and construction. Wire 82 may be detachably attachable at some point along its length by means of a coupling 86. The wire 82 may then be referred to as having two portions 82a, 82b corresponding to the wire leading to the hydrophone and the wire leading to the playback unit, respectively.

The hydrophone wire 82a passes through the wall of the bladder 72 and, in doing so, must pass through a sealed juncture 88 to ensure that no fluid leaks out past the point where the wire enters into the bladder 72. The hydrophone 80 is an electroacoustic device that converts electrical signals into acoustic impulses. The hydrophone 80 may convert an electronic signal into a means that is best transmitted into a fluid environment such as that present in surrounding warm tap water and other fluids.

If the hydrophone 80 and wire 82a are constructed so as to withstand microwave radiation and if the bladder 72 is likewise constructed to withstand microwaves, the neck 76 and removable cap 74 may be eliminated from the construction; and a fluid which absorbs microwaves to produce heat for a long period of time may be substituted for warm tap water or the like so that the sealed bladder 72 may be placed into a microwave oven and heated for a short period of time to provide a long-lasting source of fluid heat for the blanket 10 of the present invention. Precaution must be taken not to overradiate the bladder 72 and its contents with microwaves as catastrophic bursting may occur should the fluid expand and/or gasify. The coupling 86 may be a plug-and-socket configuration or other convenient configuration for the quick and easy detachable attachment of wire 82a with wire 82b. The wire 82a may be threaded through or from the hole in the flap 56 to insert or withdraw the bladder 72 from the bladder pocket 52.

The playback unit 84 is battery powered and is preferably as small as conveniently possible while preferably providing several tens of hours of playback transmissions to the hydrophone 80. Preferably, the playback unit transmits the signal that, when converted into sound, closely resembles that of a heartbeat of a pregnant mother near her time of delivery.

This sound is that with which the infant will have great familiarity as it would with the heartbeat of the mother when held close to the mother's chest. The playback unit derives its signal from a digital recording stored in a chip or other medium 90 that may store a digital recording of a single heartbeat that is endlessly repeated by the playback unit 84 while it is switched on by switch 92. The playback unit 84 may be attached to the blanket 10 near where the right arm 22 meets the torso 20 or in some other convenient area where it would not be available to the infant lying on the blanket. It may be possible to have a hydrophone/playback unit combined into one device that may be magnetically switched through the wall of the bladder 72 so that no wire needs to pass through the wall of the bladder 72. Under such circumstances, the combined playback/hydrophone device would be attached to an inside wall of the bladder 72 with a magnetic switch located as close as possible across the bladder wall.

While the present invention has been described in terms of a sheep configuration, other friendly animals may also be used and also other textures, fabrics, materials, and placements of the bladder with its hydrophone in the present invention may be made without departing from the present invention.

In order to use the present invention, the removable cap 74 is taken off the neck 76 at the threaded aperture end of the neck 78. Lukewarm tap water may then be poured into the open neck 76 to fill the bladder 72. The hydrophone 80 will then be immersed within the lukewarm water; and upon threading the cap 74 from the neck 76, the bladder 72 may be placed in the bladder pocket 52.

Wire 82a is then threaded through aperture 58 and attached at the coupling 86 to wire 82b. The playback unit 84 is then switched on at switch 92 to transmit the heartbeat signals stored in the chip 90 to the hydrophone 80. Hydrophone 80 then converts the signals transmitted to it by the playback unit 84 into acoustic signals that are transmitted to the surrounding lukewarm water. These signals should be loud enough to be audible to one pressing their ear to the face 18 of the sheep and hopefully a little louder so that one's ear adjacent to the head 12 of the blanket 10 would notice the sound. The back 40 of the blanket is then laid upon a floor or other flat surface; and the infant, in its pajamas or otherwise, is then laid upon the front of the blanket 10. The soft, warm, and cuddly surface of the blanket 10 provides the infant with a comfortable environment; and the warmth from the water in the bladder 72 will radiate out from the head 12 or face 18 of the blanket 10. As the infant becomes accustomed to its surroundings, it will notice the heartbeat sound of the hydrophone coming from the head 12 of the blanket 10 and will be soothed and comforted until ultimately lulled into a state of restful sleep. Should the infant remain alert, the toy lamb 24 or the mirror 30 can be used to entertain the infant.

Although several embodiments have been described in detail for purposes of illustration, various modifications may be made without departing from the scope and spirit of the invention. Accordingly, the invention is not to be limited, except as by the appended claims.

What is claimed is:

1. An infant blanket for comforting an infant, the blanket comprising:
a soft outer shell for contact with the infant, said shell defining a chamber;
a bladder, said bladder fitting within said chamber, said bladder fillable with fluid and sealing said fluid within said bladder without leaking;
a hydrophone, said hydrophone residing in said bladder, said hydrophone capable of transmitting sound into said bladder when said bladder is filled with fluid; and
playback means for playing back sound, said playback means coupled to said hydrophone; whereby said bladder may be filled with fluid and sealed, then placed within said chamber, said playback means may play back sounds to be heard by the infant through said hydrophone and said bladder, said infant generally engaging said blanket adjacent said bladder, said infant comforted by the yielding nature of said fluid-filled bladder and the sounds transmitted into said bladder by said hydrophone.

2. The infant blanket for comforting an infant as set forth in claim 1, wherein said soft outer shell comprises a material selected from the group consisting of cotton, terry cloth, fleecy sheep skin, polyester, and wool.

3. The infant blanket for comforting an infant of claim 1, wherein said bladder comprises a flexible plastic bladder having a capped opening through which said fluid may flow to fill or empty said bladder.

4. The infant blanket for comforting an infant of claim 1, wherein said fluid is warm and comforting to the infant.

5. The infant blanket for comforting an infant of claim 1, wherein a wire coupled to said hydrophone passes through a wall of said bladder.

6. The infant blanket for comforting an infant of claim 5, wherein said wire may be detachably coupled to said playback means.

7. The infant blanket for comforting an infant of claim 1, wherein said playback means comprises a digitally encoded recording.

8. The infant blanket for comforting an infant of claim 7, wherein said playback means is a digital playback means and said digitally encoded recording is played continuously in a loop to continuously repeat said digitally encoded recording.

9. The infant blanket for comforting an infant of claim 8, wherein said digitally encoded recording is a recording of a heartbeat that soothes and comforts the infant.

10. The infant blanket for comforting an infant of claim 1, wherein said outer shell is in the shape of a sheep.

11. The infant blanket for comforting an infant of claim 10, further comprising a mirror, said mirror coupled to an appendage-like portion of said sheep-shaped outer shell.

12. The infant blanket for comforting an infant of claim 11, wherein said outer shell is generally flat so that the infant may lie upon the blanket.

13. The infant blanket for comforting an infant of claim 12, wherein said soft outer shell defines an opening for said chamber is present in said blanket behind a portion of said blanket corresponding to a sheep's head.

14. An infant blanket for comforting an infant, the blanket comprising:
a soft outer shell in the shape of a sheep for contact with the infant, said shell defining a chamber, said outer shell defines an opening for said chamber present in said blanket behind a portion of said blanket corresponding to a sheep's head, said outer shell being generally flat so that the infant may lie upon the blanket, said soft outer shell comprising a material selected from the group consisting of cotton, terrycloth, fleecy sheep skin, polyester, and wool;
a mirror coupled to an appendage-like portion of said sheep-shaped outer shell;
a flexible plastic bladder having a capped opening through which fluid may flow to fill or empty said bladder, said bladder fitting within said chamber, said bladder fillable with said fluid and sealing said fluid within said bladder without leaking, said fluid being warm and comforting to the infant;
a hydrophone, a wire coupled to said hydrophone passing through a wall of said bladder, said hydrophone capable of transmitting sound into said bladder when said bladder is filled with fluid; and
digital playback means for continually playing back a digitally encoded sound recording of a heartbeat that soothes and comforts the infant in a loop to continuously repeat said digitally encoded sound recording, said playback means coupled to said hydrophone, said wire detachably coupled to said playback means;
whereby said bladder may be filled with fluid and sealed, then placed within said chamber, said playback means may play back sounds to be heard by the infant through said hydrophone and said bladder, said infant generally engaging said blanket adjacent said bladder, said infant comforted by the yielding nature of said fluid-filled bladder and the sounds transmitted into said bladder by said hydrophone.
